(19) United States
(12) Patent Application Publication
(10) Pub. No.: US 2021/0267551 A1
(43) Pub. Date: Sep. 2, 2021

(54) NOISE DETECTION METHOD AND APPARATUS

(71) Applicant: ANHUI HUAMI INFORMATION TECHNOLOGY CO., LTD., Hefei (CN)

(72) Inventors: Guokang Zhu, Hefei (CN); Wei Zhao, Hefei (CN); Kongqiao Wang, Hefei (CN)

(21) Appl. No.: 16/630,564

(22) PCT Filed: Aug. 30, 2019
(86) PCT No.: PCT/CN2019/103733

§ 371 Date: Jan. 13, 2020

(30) Foreign Application Priority Data
Oct. 31, 2018 (CN) 201811286825.9

(51) Int. Cl. A61B 5/00; A61B 5/024

(52) U.S. Cl. CPC A61B 5/7207; A61B 5/02416; A61B 5/681

(57) ABSTRACT

The present disclosure provides a noise detection method and a noise detection apparatus. The method includes: segmenting a collected photoplethysmography signal into a plurality of sub-signal segments; extracting a characteristic of each sub-signal segment; for each sub-signal segment, determining a self-similarity of the sub-signal segment in the photoplethysmography signal based on the characteristic of the sub-signal segment; and determining that the sub-signal segment is noise when the self-similarity is lower than a threshold.

### CROSS REFERENCE TO RELATED APPLICATION

This application is a U.S. national phase of International Application No. PCT/CN2019/103733, which is based upon and claims a priority to Chinese Patent Application No. 201811286825.9, filed with the China National Intellectual Property Administration on Oct. 31, 2018, the entire contents of which are incorporated herein by reference.

### TECHNICAL FIELD

The present disclosure relates to the field of signal analysis, and more particularly to a noise detection method and a noise detection apparatus.

### BACKGROUND

At present, wearable devices are increasingly utilized to analyze physiological signs for health monitoring and diagnosis. The wearable device may collect photoplethysmography (PPG) signals through PPG sensors and analyze the physiological signs based on the collected PPG signals. Therefore, it can be known that a quality of PPG signals is a key factor affecting an accuracy of an analysis result of physiological signs. However, noise may be introduced during collecting the PPG signals due to interference such as skin characteristics of a wearer, a contact distance, an ambient light condition, a limb movement, which decreases the quality of PPG signals.

In the related art, in order to improve the quality of PPG signals, adaptive filtering or signal decomposition is performed on the collected PPG signals in the time domain and frequency domain, to filter out the noise in the PPG signals. However, some well-known noise (such as, high and low frequency noise or prior frequency band noise) may be filtered out by the adaptive filtering or the signal decomposition, but unknown noise may not be filtered out, which may still affect the accuracy and reliability of the analysis result of physiological signs.

### SUMMARY

According to a first aspect of embodiments of the present disclosure, a noise detection method is provided. The method includes: segmenting a collected PPG signal into a plurality of sub-signal segments; extracting a characteristic of each sub-signal segment; and for each sub-signal segment, determining a self-similarity of the sub-signal segment in the PPG signal based on the characteristic of the sub-signal segment, and determining that the sub-signal segment is noise when the self-similarity is lower than a threshold.

According to a second aspect of embodiments of the present disclosure, a wearable device is provided. The device includes: a readable storage medium and a processor. The readable storage medium is configured to store machine executable instructions. The processor is configured to read and to execute the machine executable instructions stored in the readable storage medium to implement the method according to the first aspect above.

According to a third aspect of embodiments of the present disclosure, a readable storage medium is provided. The storage medium has thereon stored one or more programs. When the one or more programs are executed by a device, the device is caused to perform the method according to the first aspect of embodiments of the present disclosure.

### BRIEF DESCRIPTION OF THE DRAWINGS

FIG. 1A is a schematic diagram illustrating a PPG signal not including noise according to an exemplary embodiment of the present disclosure.

FIG. 1B is a schematic diagram illustrating a PPG signal including noise according to an exemplary embodiment of the present disclosure.

FIG. 2A is a flow chart illustrating a noise detection method according to an exemplary embodiment of the present disclosure.

FIG. 2B is a schematic diagram illustrating a valley point-a peak point-a valley point of a sub-signal segment according to the embodiment illustrated in FIG. 2A.

FIG. 2C is a distribution diagram illustrating six-dimension characteristics before normalization according to the embodiment illustrated in FIG. 2A.

FIG. 2D is a distribution diagram illustrating six-dimension characteristics after normalization according to the embodiment illustrated in FIG. 2A.

FIG. 3 is a hardware structure diagram illustrating a wearable device according to an exemplary embodiment of the present disclosure.

FIG. 4 is a block diagram illustrating a noise detection apparatus according to an exemplary embodiment of the present disclosure.

### DETAILED DESCRIPTION

Exemplary embodiments will be described in detail herein, examples of which are illustrated in the accompanying drawings. When the following description refers to the accompanying drawings, the same or similar elements may be denoted by the same number in different accompanying drawings, unless otherwise indicated. The implementations described in the following exemplary embodiments do not represent all implementation consistent with the present disclosure. Instead, they are merely examples of apparatuses and methods consistent with aspects of the present disclosure as detailed in the appended claims.

The terminology used in the present disclosure is for the purpose of describing particular embodiments only and is not intended to be limiting of the present disclosure. As used in the description of the present disclosure and the appended claims, “a” and “the” in singular forms mean including plural forms, unless clearly indicated in the context otherwise. It should also be understood that, as used herein, the term “and/or” represents and contains any one and all possible combinations of one or more associated listed items.

It should be understood that, although the present disclosure may use terms such as “first”, “second” and “third” herein for describing various information, such information should not be limited by these terms. These terms are only used for distinguishing the same type of information from each other. For example, a first information may also be called as a second information, and similarly, the second information may also be called as the first information, without departing from the scope of the present disclosure. Depending on the context, the term “if” may be understood to mean “when” or “upon” or “in response to the determination of”.

Since removal of interference noise is an important part of photoplethysmography (PPG) signal analysis, accuracy of noise removal determines accuracy and reliability of calculation for data of human physiological signs in an actual application. At present, the PPG signal is generally used to calculate a heart rate, that is, after adaptive filtering or signal decomposition are performed on the collected PPG signal in the time domain, the heart rate is calculated according to a periodicity of the PPG signal in the frequency domain. Such calculation for the heart rate based on the frequency has a certain tolerance to the noise in the PPG signal. Therefore, some well-known noise is filtered out by the adaptive filtering or the signal decomposition.

With increasing popularity of wearable devices, a quality of a signal collected by a sensor is improved constantly, and more and more people start to employ the PPG signal to analyze a heartbeat rhythm abnormal condition in a human body (such as atrial fibrillation). Analysis for the heartbeat rhythm abnormal condition in the human body needs to accurately locate all heartbeat positions (peak positions) and amplitudes, but presence of a noise peak may directly produce a false heartbeat interval, thus affecting an accuracy judgment for the heartbeat rhythm abnormal condition. Therefore, the analysis for the heartbeat rhythm abnormal condition has a higher requirement for the quality of the PPG signal and has a lower tolerance for the noise. In the related art, a noise filtering method may not accurately remove single noise peaks and abnormal pulse peaks from the perspectives of the filtering and the signal decomposition. Therefore, the noise filtering method in the related art may not meet the application requirement for analyzing the heartbeat rhythm abnormal condition.

The PPG signal is to measure a quantity of light transmitted or reflected to a photodiode when light of a light emitting diode (LED) illuminates a human skin, to detect volume changes caused by pulse pressure. In each heartbeat cycle of the human body, a heart transmits blood to ends of the human body, and the pulse pressure causes arteries and arterioles to expand in a subcutaneous tissue, thus causing a reflectance of the human skin to light changes. Therefore, such periodic change is directly reflected in the PPG signal.

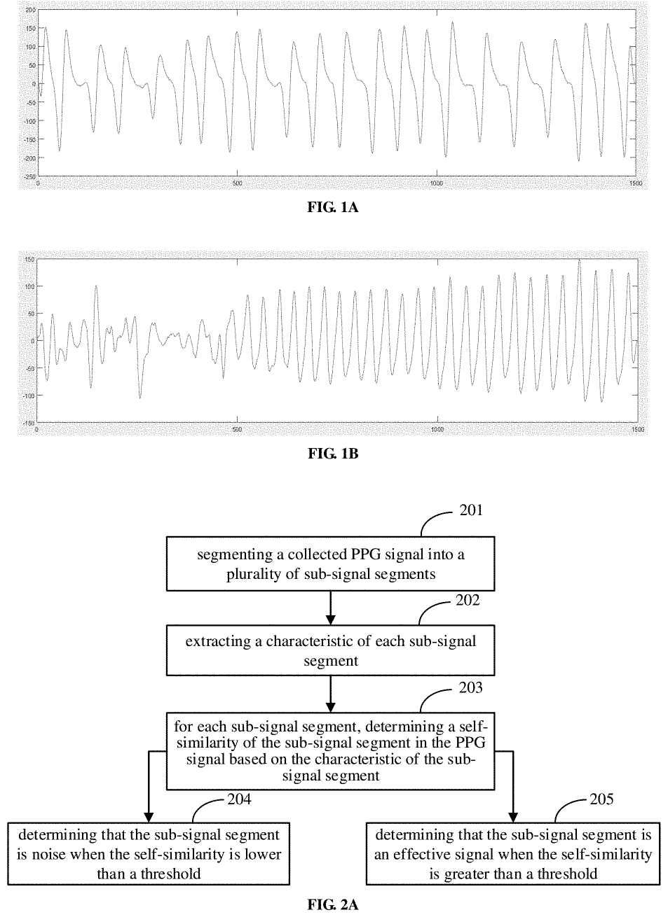

FIG. 1 is a schematic diagram illustrating an exemplary PPG signal, most of effective information of which is concentrated in peaks, and there is a need to perform an accurate location on peaks of pulses in a heartbeat rhythm analysis application. However, in an actual collection procedure, a collected PPG signal usually includes a plurality of noise peaks due to interference of skin characteristics of a wearer, a contact distance, an ambient light condition, a limb movement and other factors. For example, FIG. 1B is a schematic diagram illustrating an exemplary PPG signal including noise.

It may be known based on a difference between peaks in FIG. 1A and FIG. 1B that, peak shapes of PPG signals generated by different people are quite different due to individual differences, and the peak shapes generated by the same people in different physiological states also have great differences. However, the peak shapes generated by the same people in a shorter time period have greater self-similarities, and noise peaks generated by noise interference usually have different shapes, and especially in the shorter time period, noise peaks are characterized by extremely poor self-similarities.

Based on the above analysis, the collected PPG signal may be segmented into the plurality of sub-signal segments; the characteristic of each sub-signal segment is extracted; for each sub-signal segment, the self-similarity of the sub-signal segment in the PPG signal is determined based on the characteristic of the sub-signal segment, and it is determined that the sub-signal segment is noise when the self-similarity is lower than the threshold.

It may be known from the description above that, the collected PPG signal is segmented into the sub-signal segments, the self-similarity of each sub-signal segment in the PPG signal is determined by utilizing the characteristic of the sub-signal segment, and it is determined whether the sub-signal segment is noise based on the self-similarity. In this way, different types of noise may be detected by the self-similarity of the signal, and the noise interference is reduced. Since a signal generated by the human body in a shorter time period has a greater self-similarity, an obtained effective signal of which the self-similarity is greater than the threshold also confirms to an actual characteristic of the physiological sign, which may be used to analyze data of the physiological sign accurately. Based on the self-similarity of the signal, a plurality of different types of noise in the PPG signal may be detected, thus improving accuracy and reliability for detecting the PPG signal.

Description will be made in detail below to the technical solution of the present disclosure with detailed embodiments.

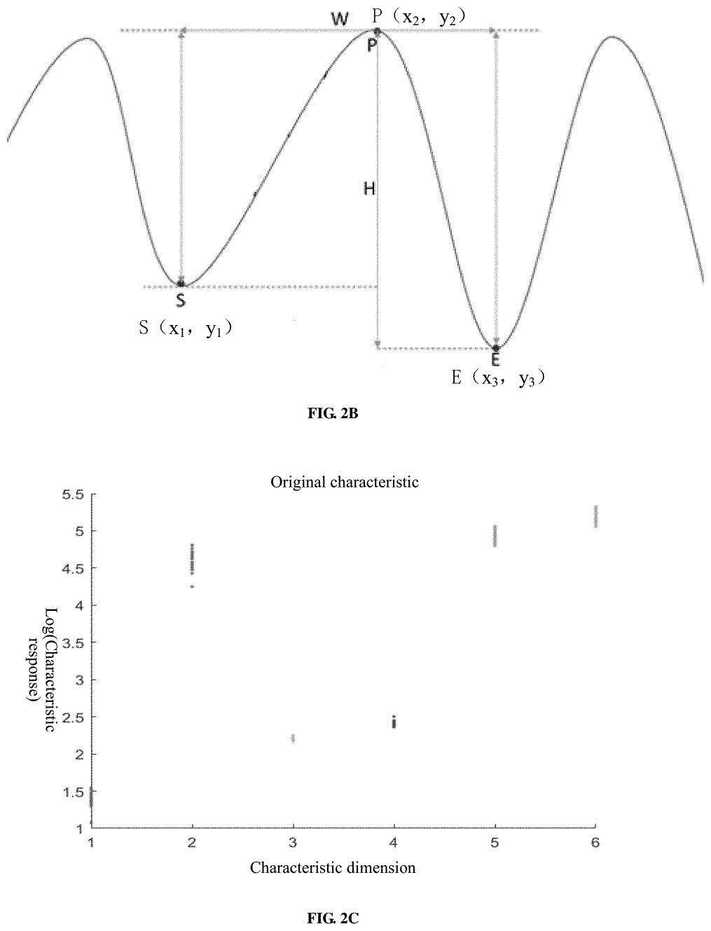

FIG. 2A is a flow chart illustrating a noise detection method according to an exemplary embodiment of the present disclosure. The noise detection method may be applied to a wearable device (e.g., a smart bracelet or a smart watch). As illustrated in FIG. 2A, the noise detection method includes actions at following blocks.

At block 201, a PPG signal is segmented into a plurality of sub-signal segments.

In an embodiment, peak shapes generated by a same people in different physiological states have greater differences, but peak shapes generated by the same people in a shorter time period have greater self-similarities. Therefore, a PPG signal in a preset time period (such as 20 seconds) may be collected for noise detection.

In an embodiment, peak points of peaks and valley points of valleys included in the PPG signal are extracted; and the PPG signal is segmented into the plurality of sub-signal segments based on the extracted peak points and the extracted valley points. Each sub-signal segment includes the same number of one or more peaks.

When the PPG signal is segmented based on the extracted peak points and the extracted valley points, each sub-signal segment may include one peak or a plurality of peaks based on experience. A segmenting point of each sub-signal segment may be located at a valley point or at the middle of a peak point and a valley point. It may ensure that each sub-signal segment includes the same number of one or more peaks. The smaller the number of one or more peaks included in the sub-signal segment, the more accurate the detection is. When each sub-signal segment only includes one peak, it is determined whether each peak in the PPG signal is an abnormal peak.

It should be noted that, the peak point of the peak included in the PPG signal may be a peak point of a significant peak. The peak of which a value of the peak point exceeds a preset value is defined as the significant peak. The preset value may be set based on actual experience.

At block 202, a characteristic of each sub-signal segment is extracted.

In an embodiment, since most of effective information of the PPG signal is concentrated at the peaks, the characteristic of each sub-signal segment may be represented by a characteristic of the included one or more peaks. Based on the segmenting way for the sub-signal segment described at block 201 above, when each sub-signal segment includes one peak, a characteristic of one peak may be used to represent the characteristic of the sub-signal segment, and when each sub-signal segment includes a plurality of peaks, in order to associate the plurality of peaks included in the sub-signal segment, characteristics of the plurality of peaks and a statistical characteristic of the characteristics of the plurality of peaks may be used to represent the characteristic of the sub-signal segment. The procedure for extracting the characteristic of the sub-signal segment may be described in two conditions below.

First condition (the sub-signal segment includes one peak): for each sub-signal segment, a morphology characteristic of the peak is determined, based on the peak point of the peak included in the sub-signal segment and valley points of two valleys neighboring to the peak, and the determined morphology characteristic is taken as the characteristic of the sub-signal segment.

The morphology characteristic of the peak may include a combination of one or more of: a peak width, a maximum drop from the peak to the valley, a peak skewness, a height ratio on both sides of the peak, gradient variances on both sides of the peak, whether there is an abnormal gradient on both sides of the peak in seven-dimension characteristics. The seven-dimension characteristics included in the above morphology characteristic are merely for exemplary illustration. The present disclosure does not define the morphology characteristic and the dimensions included by the morphology characteristic. Other morphology characteristics describing the peak may also fall within the protection scope of the present disclosure.

In an example, as illustrated in FIG. 2B, point S, point P, point E respectively correspond to one valley point, one peak point and one valley point. The coordinate of point S is (x₁, y₁), the coordinate of point P is (x₂, y₂), and the coordinate of point E is (x₃, y₃). According to this, the peak width in the morphology characteristic is represented as: W=|x₃−x₁|.

The maximum drop from the peak to the valley is represented as: H=max (|y₂−y₁|, |y₂−y₃|).

The peak skewness is represented as: R_{w}=|x₂−x₁|/W.

The height ratio on both sides of the peak is represented as: R_{H}=|y₂−y₁|/|y₂−y₃|.

The skilled in the art should be understood that, the peak skewness may also be represented as: R_{w}=|x₃−x₂|/W; and the height ratio on both sides of the peak may also be represented as R_{H}=|y₂−y₁|/|y₂−y₃|.

A gradient variance on the left of the peak (an increasing gradient variance) is represented as: V_{S,P}=var(PPG′(S,P)). PPG′(S,P) represents a first-order difference from point S to point P, and var( ) represents a variance.

A gradient variance on the right of the peak (a decreasing gradient variance) is represented as: V_{P,E}=var(PPG′(P,E)). PPG′(P,E) represents a first-order difference from point P to point E, and var( ) represents a variance.

Whether there is an abnormal gradient on both sides of the peak is represented as: Z_{S,P,E}[Σ(PPG′(S,P)<0)> a preset value]|[Σ(PPG′(P,E)>0)> a preset value]. Σ(PPG′(S,P)<0) represents the number of points on the left of the peak of which gradients are lower than 0. Σ(PPG′(P,E)>0) represents the number of points on the right of the peak of which gradients are greater than 0. When a value of Z_{S,P,E} is 1, it represents that there is the abnormal gradient, and when a value of Z_{S,P,E} is 0, it represents that there is not the abnormal gradient. The preset value may be set according to actual experience. For example, the preset value is set as 5. It is represented that, when the number of points on the left of the peak of which gradients are lower than 0 is more than 5, or the number of points on the right of the peak of which gradients are greater than 0 is more than 5, Z_{S,P,E}=1, and it is determined that there are abnormal gradients on both sides of the peak.

A second condition (the sub-signal segment includes the plurality of peaks): for each sub-signal segment, morphology characteristics of respective peaks is determined based on peak points of respective peaks included in the sub-signal segment and valley points of two valleys neighboring to each peak, a statistical characteristic is calculated by utilizing the morphology characteristics of respective peaks, and the morphology characteristics of respective peaks and the statistical characteristic is taken as the characteristic of the sub-signal segment.

The procedure for determining the morphology characteristics of respective peaks may be referred to content described in the first condition. The statistical characteristic may be an average morphological characteristics (including an average value of the peak widths, an average value of maximum drops from the peaks to the valleys, an average value of peak skewnesses, an average value of height ratios on both sides of the peaks, an average value of gradient variances on both sides of the peaks) of the morphological characteristics of respective peaks, or a mid-value morphological characteristic (including a mid-value of the peak widths, a mid-value of maximum drops from the peaks to the valleys, a mid-value of peak skewnesses, a mid-value of height ratios on both sides of the peaks, a mid-value of gradient variances on both sides of the peaks) of the morphological characteristics of respective peaks

The skilled in the art may understood that, the present disclosure takes that the morphological characteristic of the peak represents the characteristic of the sub-signal segment as an example for description, but in addition to the morphological characteristic, other characteristics of the peak (such as, a time-frequency domain characteristic of the peak) may be used to represent the characteristic of the sub-signal segment, which is not limited by the present disclosure. The method for using other characteristics of the peak to represent the characteristic of the sub-signal segment may also fall with the protection scope of the present disclosure.

It should be noted that, since the peak width, the maximum drop from the peak to the valley, the peak skewness, the height ratio on both sides of the peak in the above determined morphology characteristic are different dimensions, in order to facilitate subsequent self-similarity calculation, there is a need to normalize respective characteristics in the morphological characteristics to a uniform dimension. Based on this, after the characteristic of each sub-signal segment is extracted, the normalization may also performed on the peak width, the maximum drop from the peak to the valley, the peak skewness, the height ratio on both sides of the peak, and the gradient variances on both sides of the peak, included in the characteristic of each sub-signal segment.

An equation of the normalization may be

$${f}_{j}^{′} ( {g}_{i} ) = \frac{{f}_{j} ( {g}_{i} )}{\frac{1}{n} {\sum }_{k = 1}^{n} {f}_{j} ( {g}_{k} )} , j ∈ [ 0 , 6 ) .$$

The PPG signal is segmented into a plurality of segments represented as G={g₁, g₂ . . . gₙ}. n represents the number of the sub-signal segments. j represents the j^{-th} dimension characteristic (performing the normalization on the above six-dimension characteristics). gᵢ represents the i^{-th} sub-signal segment. fⱼ(gᵢ) represents a characteristic value of the j^{-th} dimension characteristic in the i^{-th} sub-signal segment. fⱼ′(gᵢ) represents a normalization characteristic value of the j^{-th} dimension characteristic in the i^{-th} sub-signal segment.

In an example, as illustrated in FIGS. 2C-2D, FIG. 2C is a distribution diagram illustrating six-dimension characteristics before the normalization in the plurality of sub-signal segments. Since a span of a characteristic response of each dimension characteristic is large, the characteristic response is marked on the vertical axis after taking the log. FIG. 2D is a distribution diagram illustrating six-dimension characteristics after normalization in the plurality of sub-signal segments. After the normalization is performed, the dimensions of respective characteristics are uniform, and the span of the characteristic response is smaller, which may be concentrated within a certain value range.

At block 203, for each sub-signal segment, a self-similarity of the sub-signal segment in the PPG signal is determined based on the characteristic of the sub-signal segment.

In an embodiment, in a signal processing field, a known characteristic of the signal (a priori knowledge) have a great implication for signal analysis. In detail, the PPG signal effectively reflects a relative regularity of the physiological sign, that is, it has a high probability of repeated occurrence in a certain time period. On the contrary, the noise has an unrelated characteristic. Therefore, diversified noise data would violate the self-similarity prior, and for each sub-signal segment, it may be determined whether the sub-signal segment belongs to the noise by calculating the self-similarity of the sub-signal segment.

An equation for determining the self-similarity of the sub-signal segment may be: S(gᵢ|G)=E(d(f′(gᵢ), f′(gⱼ))), gⱼ∈G. The PPG signal is segmented into n sub-signal segments represented as G={g₁, g₂ . . . gₙ}. represents the number of the sub-signal segments. f′(gᵢ) represents a characteristic description for the sub-signal gᵢ after the normalization. d( ) represents a similarity metric function. The larger a value of the similarity metric function d( ), the more similar gᵢ and gⱼ are. E( ) represents a self-similarity of gᵢ on the G, the statistic way of which may be an average statistic way, a mid-value statistic way and the like.

An equation for determining the self-similarity of the sub-signal segment may also be: S(gᵢ|G)=Σⱼ₌₁ⁿ[d′(f′(gᵢ), f′(gⱼ))<ε], gⱼ∈G. d′( ) represents an Euclidean distance measurement function. The smaller a value of the Euclidean distance measurement function d′( ), the more similar gᵢ and gⱼ are. ε represents a tolerance. Σⱼ₌₁ⁿ[ ] represents the number of the values of the Euclidean distance measurement function which are smaller than ε. The larger the number, the more similar gᵢ and gⱼ are.

It should be noted that, the self-similarity of the sub-signal segment is determined by calculation based on similarities of the characteristic of the sub-signal segment and characteristics of other sub-signal segments (including non-neighboring sub-signal segments). Therefore, the determined self-similarity of the sub-signal segment belongs to non-local self-similarities, which conforms to a relative regularity of the PPG signal, i.e., a high recurring probability characteristic in a certain time period.

At block 204, it is determined that the sub-signal segment is noise when the self-similarity is lower than a threshold.

At block 205, it is determined that the sub-signal segment is an effective signal when the self-similarity is greater than the threshold.

In an embodiment, when it is determined that the sub-signal segment is noise, the sub-signal segment is labeled as noise, and when it is determined that the sub-signal segment is the effective signal, the sub-signal segment may be labeled as efficiency, so as to facilitate a subsequent selection for effective signals.

In embodiments of the present disclosure, the collected PPG signal is segmented into the plurality of sub-signal segments; the characteristic of each sub-signal segment is extracted; for each sub-signal segment, the self-similarity of the sub-signal segment in the PPG signal is determined based on the characteristic of the sub-signal segment; and it is determined that the sub-signal segment is noise when the self-similarity is lower than the threshold.

It may be known from above that, the collected PPG signal is segmented into the sub-signal segments, the self-similarity of each sub-signal segment in the PPG signal is determined based on the characteristic of the sub-signal segment, and it is determined whether the sub-signal segment is noise based on the self-similarity. In this way, different types of noise may be detected by the self-similarity of the sub-signal segment, and the noise interference is reduced. Since a signal generated by the human body in a shorter time period has a greater self-similarity, an obtained effective signal of which the self-similarity is greater than the threshold also confirms to an actual characteristic of the physiological sign, which may be used to analyze data of the physiological sign accurately. Based on the self-similarity of the signal, a plurality of different types of noise in the PPG signal may be detected, thus improving accuracy and reliability for detecting the PPG signal.

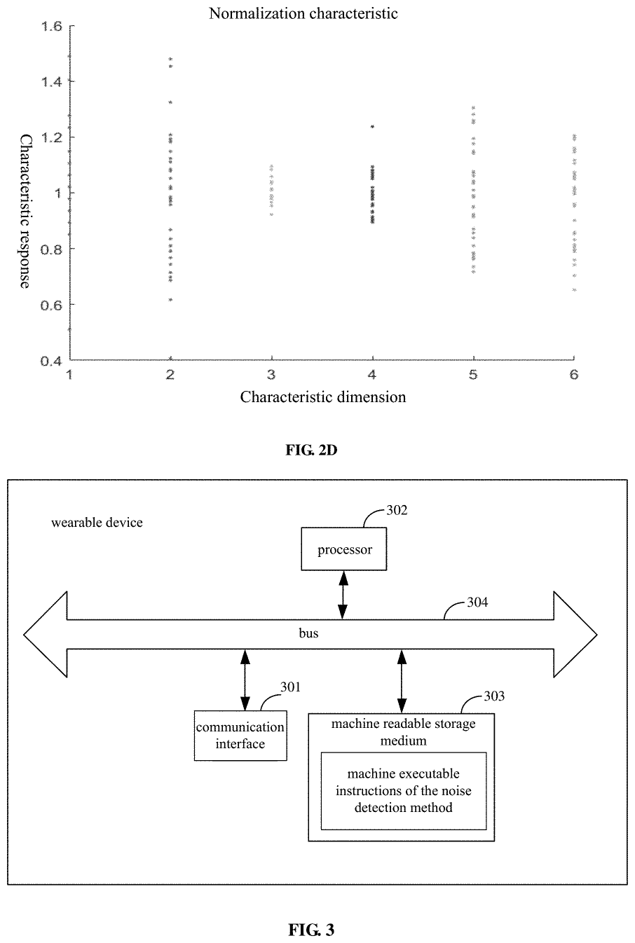

FIG. 3 is a hardware structure diagram illustrating a wearable device according to an exemplary embodiment of the present disclosure. The wearable device includes: a communication interface 301, a processor 302, a machine readable storage medium 303, and a bus 304. The communication interface 301, the processor 302, and the machine readable storage medium 303 may communicate with each other via the bus 304. The processor 302 may execute the noise detection method described above by reading and executing machine executable instructions corresponding to a control logic of the noise detection method in the machine readable storage medium 303. Detailed content of the method may be referred to the above embodiments, which is not elaborated herein.

The machine readable storage medium 303 mentioned in the present disclosure may be any electronic, magnetic, optical, or other physical storage device, and may include or store information such as executable instructions and data. For example, the machine readable storage medium may be a volatile memory, a non-volatile memory or a similar storage media. In detail, the machine readable storage medium 303 may be a random access memory (RAM), a flash, a storage drive (such as a hard drive), any type of storage disk (such as an optical disc, a digital versatile disc (DVD)), a similar storage medium, or a combination thereof.

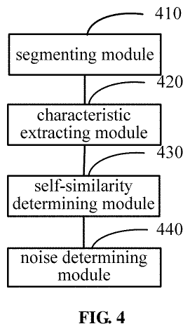

FIG. 4 is a block diagram illustrating a noise detection apparatus according to an exemplary embodiment of the present disclosure. The noise detection apparatus may be applied to a wearable device. As illustrated in FIG. 4, the noise detection apparatus includes: a segmenting module 410, a characteristic extracting module 420, a self-similarity determining module 430, and a noise determining module 440.

The segmenting module 410 is configured to segment a PPG signal into a plurality of sub-signal segments.

The characteristic extracting module 420 is configured to extract a characteristic of each sub-signal segment.

The self-similarity determining module 430 is configured to, for each sub-signal segment, determine a self-similarity of the sub-signal segment in the PPG signal based on the characteristic of the sub-signal segment.

The noise determining module 440 is configured to determine that the sub-signal segment is noise when the self-similarity is lower than a threshold. In an alternative implementation, the segmenting module 410 is configured to extract peak points of peaks and valley points of valleys included in the PPG signal; and to segment the PPG signal into the plurality of sub-signal segments based on the peak points extracted and the valley points extracted. Each sub-signal segment includes the same number of one or more peaks.

In an alternative implementation, the characteristic extracting module 420 is configured to, when each sub-signal segment includes one peak, for each sub-signal segment, determine a morphology characteristic of the peak based on the peak point of the peak included by the sub-signal segment and valley points of two valleys neighboring to the peak, and take the morphology characteristic determined as the characteristic of the sub-signal segment; and when each sub-signal segment includes two or more peaks, for each sub-signal segment, determine morphology characteristics of respective peaks based on peak points of respective peaks included by the sub-signal segment and valley points of two valleys neighboring to each peak, calculate a statistical characteristic by utilizing the morphology characteristics of respective peaks, and take the morphology characteristics of respective peaks and the statistical characteristic as the characteristic of the sub-signal segment.

In an alternative implementation, the morphology characteristic include a combination of one or more of: a peak width, a maximum drop from the peak to the valley, a peak skewness, a height ratio on both sides of the peak, gradient variances on both sides of the peak, and whether there is an abnormal gradient on both sides of the peak.

In an alternative implementation, the apparatus further includes (not illustrated in FIG. 4): a normalization module.

The normalization module is configured to, before determining the self-similarity of the sub-signal segment in the PPG signal based on the characteristic of the sub-signal segment, perform normalization processing on the peak width, the maximum drop from the peak to the valley, the peak skewness, the height ratio on both sides of the peak, and the gradient variances on both sides of the peak, included in the characteristic of each sub-signal segment.

The implementation procedure of functions of respective units in the above apparatus may be referred to the implementation procedure of the corresponding blocks in the above method, which is not elaborated herein.

For embodiments of the apparatus, since they basically correspond to the embodiments of the method, the relevant parts may be referred to the description of the embodiments of the method. The embodiments of the apparatus described above are merely illustrative. The units described as separate components may or may not be physically separate, and the component displayed as a unit may or may not be a physical unit, that is, may be located in one position, or may also be distributed in a plurality of network units. Parts or all modules of the apparatus may be selected based on an actual need to implement the objective of the present disclosure. The skilled in the art may understand and implement the method without any creative effort.

Other embodiments of the present disclosure will be apparent to those skilled in the art from consideration of the specification and practice of the present disclosure disclosed here. This application is intended to cover any variations, uses, or adaptations of the present disclosure following the general principles thereof and including such departures from the present disclosure as come within known or customary practice in the art. It is intended that the specification and embodiments may be considered as exemplary only, with a true scope and spirit of the present disclosure being indicated by the following claims.

It further should be noted that, terms “including”, “comprising” or any other variant thereof are intended to cover a non-exclusive inclusion, such that procedures, methods, products or devices including a series of elements not only include those elements, but also include other elements not explicitly listed, or further includes elements inherent in such procedures, methods, products, or devices. Without more restrictions, sentence “includes one . . . ” defines one element, but it is not excluded that there are additional identical elements in the procedures, methods, products, or devices that includes the element.

The above description is only the optimum embodiments of the present disclosure, which is not used to limit the present disclosure. Any modification, equivalent replacement, improvement and the like made within the spirit and principle of the present disclosure shall be regarded as within the protection scope of the present disclosure.

1. A noise detection method, comprising:
segmenting a collected photoplethysmography signal into a plurality of sub-signal segments;
extracting a characteristic of each sub-signal segment; and
for each sub-signal segment, determining a self-similarity of the sub-signal segment in the photoplethysmography signal based on the characteristic of the sub-signal segment, and determining that the sub-signal segment is noise when the self-similarity is lower than a threshold.

2. The method of claim 1, wherein, segmenting the photoplethysmography signal into the plurality of sub-signal segments comprises:
extracting peak points of peaks and valley points of valleys comprised in the photoplethysmography signal; and
segmenting the photoplethysmography signal into the plurality of sub-signal segments based on the peak points extracted and the valley points extracted; each sub-signal segment comprising the same number of one or more peaks.

3. The method of claim 1, wherein, extracting the characteristic of each sub-signal segment comprises:
when each sub-signal segment comprises one peak, for each sub-signal segment, determining a morphology characteristic of the peak, based on the peak point of the peak comprised in the sub-signal segment and valley points of two valleys neighboring to the peak, and taking the morphology characteristic determined as the characteristic of the sub-signal segment; and
when each sub-signal segment comprises two or more peaks, for each sub-signal segment, determining morphology characteristics of respective peaks based on peak points of respective peaks comprised in the sub-signal segment and valley points of two valleys neighboring to each peak, calculating a statistical characteristic by utilizing the morphology characteristics of respective peaks, and taking the morphology characteristics of respective peaks and the statistical characteristic as the characteristic of the sub-signal segment.

4. The method of claim 3, wherein, the morphology characteristic comprises a combination of one or more of: a peak width, a maximum drop from the peak to the valley, a peak skewness, a height ratio on both sides of the peak, gradient variances on both sides of the peak, and whether there is an abnormal gradient on both sides of the peak.

5. The method of claim 1, further comprising:
performing normalization processing on the peak width, the maximum drop from the peak to the valley, the peak skewness, the height ratio on both sides of the peak, and the gradient variances on both sides of the peak, comprised in the characteristic of each sub-signal segment.

6.-10. (canceled)

11. A wearable device, comprising:
a readable storage medium, configured to store machine executable instructions; and
a processor, configured to read and to execute the machine executable instructions stored in the readable storage medium to implement a method comprising:
segmenting a collected photoplethysmography signal into a plurality of sub-signal segments;
extracting a characteristic of each sub-signal segment; and
for each sub-signal segment, determining a self-similarity of the sub-signal segment in the photoplethysmography signal based on the characteristic of the sub-signal segment, and determining that the sub-signal segment is noise when the self-similarity is lower than a threshold.

12. The method of claim 1, further comprising:
skipping a peak having a peak point not exceeding a preset value, in the photoplethysmography signal when segmenting the collected photoplethysmography signal.

13. The method of claim 1, wherein a segmenting point of each sub-signal segment is located at a valley point or at a middle of a peak point and a valley point.

14. The wearable device of claim 11, wherein, segmenting the photoplethysmography signal into the plurality of sub-signal segments comprises:
extracting peak points of peaks and valley points of valleys comprised in the photoplethysmography signal; and
segmenting the photoplethysmography signal into the plurality of sub-signal segments based on the peak points extracted and the valley points extracted; each sub-signal segment comprising the same number of one or more peaks.

15. The wearable device of claim 11, wherein, extracting the characteristic of each sub-signal segment comprises:
when each sub-signal segment comprises one peak, for each sub-signal segment, determining a morphology characteristic of the peak, based on the peak point of the peak comprised in the sub-signal segment and valley points of two valleys neighboring to the peak, and taking the morphology characteristic determined as the characteristic of the sub-signal segment; and
when each sub-signal segment comprises two or more peaks, for each sub-signal segment, determining morphology characteristics of respective peaks based on peak points of respective peaks comprised in the sub-signal segment and valley points of two valleys neighboring to each peak, calculating a statistical characteristic by utilizing the morphology characteristics of respective peaks, and taking the morphology characteristics of respective peaks and the statistical characteristic as the characteristic of the sub-signal segment.

16. The wearable device of claim 15, wherein, the morphology characteristic comprises a combination of one or more of: a peak width, a maximum drop from the peak to the valley, a peak skewness, a height ratio on both sides of the peak, gradient variances on both sides of the peak, and whether there is an abnormal gradient on both sides of the peak.

17. The wearable device of claim 11, wherein the method further comprises:
performing normalization processing on the peak width, the maximum drop from the peak to the valley, the peak skewness, the height ratio on both sides of the peak, and the gradient variances on both sides of the peak, comprised in the characteristic of each sub-signal segment.

18. The wearable device of claim 11, wherein the method further comprises:
skipping a peak having a peak point not exceeding a preset value, in the photoplethysmography signal when segmenting the collected photoplethysmography signal.

19. The wearable device of claim 11, wherein a segmenting point of each sub-signal segment is located at a valley point or at a middle of a peak point and a valley point.

20. A non-transitory computer-readable storage medium having stored therein instructions that, when executed by a processor of a device, causes the device to perform a method comprising:
segmenting a collected photoplethysmography signal into a plurality of sub-signal segments;
extracting a characteristic of each sub-signal segment; and
for each sub-signal segment, determining a self-similarity of the sub-signal segment in the photoplethysmography signal based on the characteristic of the sub-signal segment, and determining that the sub-signal segment is noise when the self-similarity is lower than a threshold.

21. The non-transitory computer-readable storage medium of claim 20, wherein, segmenting the photoplethysmography signal into the plurality of sub-signal segments comprises:
extracting peak points of peaks and valley points of valleys comprised in the photoplethysmography signal; and
segmenting the photoplethysmography signal into the plurality of sub-signal segments based on the peak points extracted and the valley points extracted; each sub-signal segment comprising the same number of one or more peaks.

22. The non-transitory computer-readable storage medium of claim 20, wherein, extracting the characteristic of each sub-signal segment comprises:
when each sub-signal segment comprises one peak, for each sub-signal segment, determining a morphology characteristic of the peak, based on the peak point of the peak comprised in the sub-signal segment and valley points of two valleys neighboring to the peak, and taking the morphology characteristic determined as the characteristic of the sub-signal segment; and
when each sub-signal segment comprises two or more peaks, for each sub-signal segment, determining morphology characteristics of respective peaks based on peak points of respective peaks comprised in the sub-signal segment and valley points of two valleys neighboring to each peak, calculating a statistical characteristic by utilizing the morphology characteristics of respective peaks, and taking the morphology characteristics of respective peaks and the statistical characteristic as the characteristic of the sub-signal segment.

23. The non-transitory computer-readable storage medium of claim 22, wherein, the morphology characteristic comprises a combination of one or more of: a peak width, a maximum drop from the peak to the valley, a peak skewness, a height ratio on both sides of the peak, gradient variances on both sides of the peak, and whether there is an abnormal gradient on both sides of the peak.

24. The non-transitory computer-readable storage medium of claim 20, wherein the method further comprises:
performing normalization processing on the peak width, the maximum drop from the peak to the valley, the peak skewness, the height ratio on both sides of the peak, and the gradient variances on both sides of the peak, comprised in the characteristic of each sub-signal segment.

25. The non-transitory computer-readable storage medium of claim 20, wherein the method further comprises:
skipping a peak having a peak point not exceeding a preset value, in the photoplethysmography signal when segmenting the collected photoplethysmography signal.
